(19) United States
(12) Patent Application Publication
(10) Pub. No.: US 2025/0135157 A1
(43) Pub. Date: May 1, 2025

(54) USER INTERFACE AND GUI FOR SEPARATION OF TRANSLATION AND ROTATION MOTIONS FOR ROBOTIC CATHETER APPLICATION

(71) Applicant: Covidien LP, Mansfield, MA (US)

(72) Inventors: Geoffrey A. Orth, Sebastopol, CA (US); Dany Junio, Brookline, MA (US); Julie A. Benton, Santa Rosa, CA (US); Robert H. White, Roslindale, MA (US); Jonathan Scott Thomson, Stoneham, MA (US)

(73) Assignee: Covidien LP, Mansfield, MA (US)

(21) Appl. No.: 18/916,515

(22) Filed: Oct. 15, 2024

Related U.S. Application Data
(60) Provisional application No. 63/546,408, filed on Oct. 30, 2023.

(51) Int. Cl. A61M 25/01; A61F 2/24

(52) U.S. Cl. CPC A61M 25/0113; A61F 2/2427

(57) ABSTRACT

A system for navigation of a luminal network including a catheter with a distal portion configured for articulation and a handle, a cradle configured to receive and support the handle, a first motor connected to the catheter, wherein the motor is operable to articulate the distal portion of the catheter, a second motor connected to the catheter, wherein the motor is operable to rotate the catheter about its longitudinal axis, and a user interface including at least one input, wherein signals generated by manipulation of the input are delivered to the first motor to articulate the catheter and to the second motor to rotate the catheter.

### FIELD

This disclosure relates to surgical systems and methods for treating tissue. More particularly, the present disclosure relates to systems and methods for controlling aspects of motor driven and robotic catheter devices.

### BACKGROUND

More and more surgeons are utilizing surgical robots to drive endoscopes, endoscopic instruments, and other catheter-based devices providing access to organs through natural orifices or small incisions. During procedures poor visibility, lack of tactile response, difficult anatomical navigation, challenges in analyzing intra-procedural images, and/or inadequate control of the instrumentation being utilized to perform the procedure all work to increase the challenges associated with these procedures. This disclosure is directed to addressing the shortcomings of prior systems.

### SUMMARY

One aspect of the disclosure is directed to a system for navigation of a luminal network. The system includes a catheter with a distal portion configured for articulation and a handle; a cradle configured to receive and support the handle. The system also includes a first motor connected to the catheter, where the motor is operable to articulate the distal portion of the catheter; a second motor connected to the catheter, where the motor is operable to rotate the catheter about its longitudinal axis; and a user interface including at least one input, where signals generated by manipulation of the input are delivered to the first motor to articulate the catheter and to the second motor to rotate the catheter. Other embodiments of this aspect include corresponding computer systems, apparatus, and computer programs recorded on one or more computer storage devices, each configured to perform the actions of the methods and systems described herein.

Implementations of this aspect of the disclosure may include one or more of the following features. The system for navigation where the cradle includes at least two supports configured to receive the catheter. The system for navigation further includes a housing secured to one of the at least two supports, the housing including a shaft connected to the first motor, the shaft including a pinion gear. The system for navigation further includes a ring gear secured to a flex controller, the flex controller operable to cause the distal portion of the catheter to articulate. Rotation of the flex controller articulates the distal portion of the catheter. The ring gear mates with the pinion gear such that rotation of the shaft and pinion gear results in rotation of the ring gear and the flex controller. The pinion gear is a worm gear. The system for navigation further includes a carrier including a shaft configured to mate with the second motor, and a pair of clamps securing the carrier to the catheter. The ring gear mates with a pinion gear on the shaft and rotation of the shaft by the motor results in rotation of the ring gear and the catheter rotation knob. The catheter includes an inner catheter, an outer catheter and a capsule catheter. The system of navigation further includes a third motor configured to mate with the cradle and effectuate longitudinal translation of the inner catheter relative to the outer catheter. The system of navigation further includes a fourth motor configured to mate with the cradle and effectuate longitudinal translation of the capsule catheter relative to the inner catheter and the outer catheter. The system for navigation further includes a motor controller configured to receive signals from the user interface and output signals to drive the first, second, third, and fourth motors. The display is configured to present a graphic user interface (GUI). The user interface is configured to receive images from one or more imaging modalities and display the images on the GUI. The system for navigation where the catheter is a nested catheter a first catheter inside of a second catheter. The first catheter is connected to the first motor to articulate the distal portion of the first catheter. The first catheter is connected to the second motor to rotate the first catheter about its longitudinal axis. The second catheter is connected to a third motor to articulate the distal portion of the second catheter. The second catheter is connected to a fourth motor to rotate the second catheter about its longitudinal axis.

Implementations of the described techniques may include hardware, a method or process, or computer software on a computer-accessible medium, including software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

### BRIEF DESCRIPTION OF THE DRAWINGS

FIG. 1 depicts a catheter system in accordance with the disclosure;

FIG. 2 depicts the catheter system of FIG. 1 in a cradle in accordance with the disclosure;

FIGS. 3A and 3B depict a drive mechanism for the catheter system of FIG. 1 in accordance with the disclosure;

FIG. 4 depicts a cradle with a plurality of drive mechanisms connected thereto in accordance with the disclosure;

FIG. 5 depicts the cradle with a plurality of drive mechanisms connected thereto in accordance with the disclosure;

FIG. 6 is a top view of the catheter system of FIG. 1 in the cradle of FIG. 2 with a plurality of drive mechanisms connected thereto in accordance with the disclosure;

FIG. 7 depicts a top view of a portion of the catheter system of FIG. 1 with drive mechanisms connected thereto in accordance with the disclosure; and

FIG. 8 depicts a system for driving the catheter of FIG. 1 in accordance with the disclosure.

### DETAILED DESCRIPTION

This disclosure is directed to systems and methods of motorizing or robotizing a catheter-based system and providing a user interface enabling finite control of the catheter system for controlled navigation within a patient. The user interface includes a graphic user interface (GUI) presenting a variety of data to the surgeon for use during the navigation of the catheter within the patient and deploying one or more fixtures, acquiring biopsies, and applying therapies to tissues of the patient.

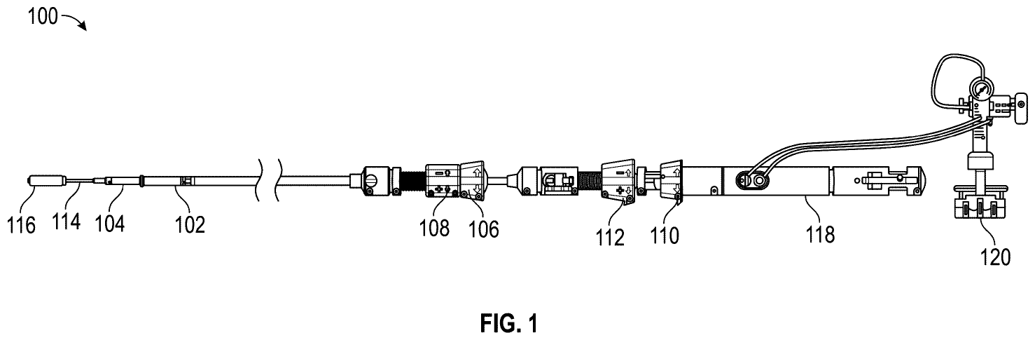

FIG. 1 depicts a catheter-based system 100. The catheter system 100 is particularly designed for navigation through the femoral vein of a patent and for placement of one or more valves, for example a replacement mitral valve as part of a Transcatheter Mitral Valve Replacement (TMVR) procedure. Details of the catheter system 100 may be found in co-pending U.S. Provisional Patent Application No. 63/505,836 titled TRANSCATHETER VALVE DELIVERY SYSTEM WITH A TELESCOPIC CAPSULE ASSEMBLY AND METHODS OF USE THEREOF, filed Jun. 2, 2023, the entire contents of which is incorporated herein by reference. While the following disclosure is focused on the catheter system 100, the disclosure is not so limited, and the methods and devices described herein can be utilized in a variety of other catheter-based systems. For example, the methods and devices described herein may be employed in conjunction with the EDGE catheter system employed for navigation of the of the lungs utilizing the ILLUMISITE lung navigation platform without departing from the scope of the disclosure. Details of the ILLUMISITE system may be found in U.S. Pat. No. 11,026,644 titled SYSTEM AND METHOD FOR NAVIGATING WITH THE LUNG, filed Sep. 25, 2023, the entire contents of which are incorporated herein by reference. The methods and devices described herein may also be employed with other lung navigation systems, in cardiac rhythm management procedures (e.g., introduction of a pacemaker), in cryoablation procedures (e.g., to treat atrial fibrillation), and other systems and procedures for delivering valves or other devices, or treatment/therapy.

The catheter-based system 100 includes an outer guide catheter 102 and an inner catheter 104 which traverses a lumen in the outer guide catheter 102. Both the outer guide catheter 102 and the inner catheter 104 are articulable or bendable in at least one direction each. In addition, both the outer guide catheter 102 and inner catheter can be independently rotated. An outer guide catheter rotation knob 106 allows for the outer guide catheter 102 to be rotated about its longitudinal axis. A flex controller 108 acts to bend or articulate the outer guide catheter 102 in at least one direction. Similarly an inner catheter rotation knob 110 allows for rotation of the inner catheter 104 while a flex controller 112 acts to bend or articulate a portion of the inner catheter 104 in at least one direction. A capsule catheter 114 including a capsule 116 at its distal end for transporting and deploying the replacement valve, rides within and may be extended from a distal portion of the inner catheter 104, by advancement of a handle portion 118 of the catheter system 100 relative to inner catheter flex controller 112. A pneumatic hand pump 120 is operatively connected to the capsule 116 to deploy the valve at the appropriate location within the patient.

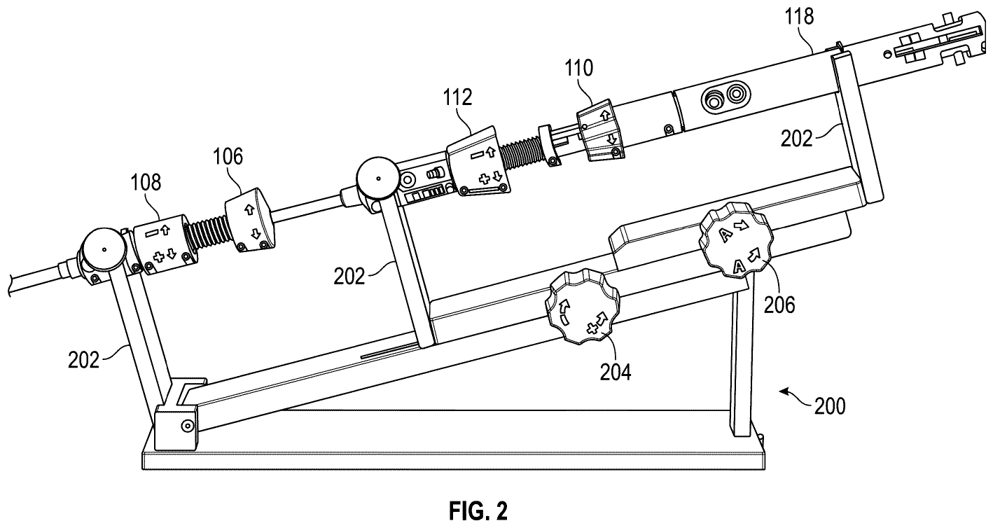

FIG. 2 depicts the catheter system 100 in a cradle 200. The cradle 200 includes three supports 202. The supports 202 hold the catheter system 100 at specified locations, secure the catheter system 100 to the cradle 200 and allow for relative motion of portions of the catheter system 100. An inner catheter drive knob 204 advances the inner catheter 104 such that the inner catheter 104 extends beyond the outer guide catheter 102. A distal support 202 proximate outer catheter flex controller 108 holds the outer guide catheter 102 in place while the inner catheter 104 and handle 118 supported by the middle support 202 and proximal support 202 are advanced in the direction of the outer catheter rotation knob 106. A capsule catheter drive knob 206 advances the capsule catheter 114 and capsule 116 from the inner catheter 104 by advancing the handle 118 supported by the proximal support 202 in the direction of the inner catheter rotation knob 110, which is prevented from moving by the middle support 202. Though depicted with three supports 202, the disclosure is not so limited and more or fewer supports 202, even just a single support may be employed without departing from the scope of the disclosure.

By manipulating the various knobs described herein above the capsule 116 and the replacement valve it carries as a payload can be navigated through a tortuous luminal network (e.g., the vascular system leading the left ventricle from the femoral vein). In a similar manner a catheter can be navigated through the airways of a patient to a target such as a tumor for biopsy or treatment. However, as will be appreciated the cradle 200 described above has two knobs for adjusting aspects of the catheter system 100 and the catheter system 100 has another four adjustment points. Thus utilizing the catheter system 100 and cradle 200 via manual manipulation can be an arduous and time-consuming task, where each sequential movement may require multiple manual manipulations just to advance a few centimeters within the patient. Making this more challenging, each surgeon has just two hands, and therefore must both manage the movements of these six adjustment points in proper sequence and without mistake to ensure proper navigation. Further, at each point where a manipulation must be made imaging (e.g., fluoroscopy or ultrasound) must be acquired to ensure that undesirable contact with tissues such as the wall of the ventricle is minimized. Each of these steps prolongs a procedure, takes up resources of multiple personnel, sometimes as many as 15 for a single procedure, and by extending the procedure adds stress and challenges to already compromised patients. Accordingly, there is a need to automate the drive and manipulation of the catheter system 100 and to provide a user interface that has a single point of contact.

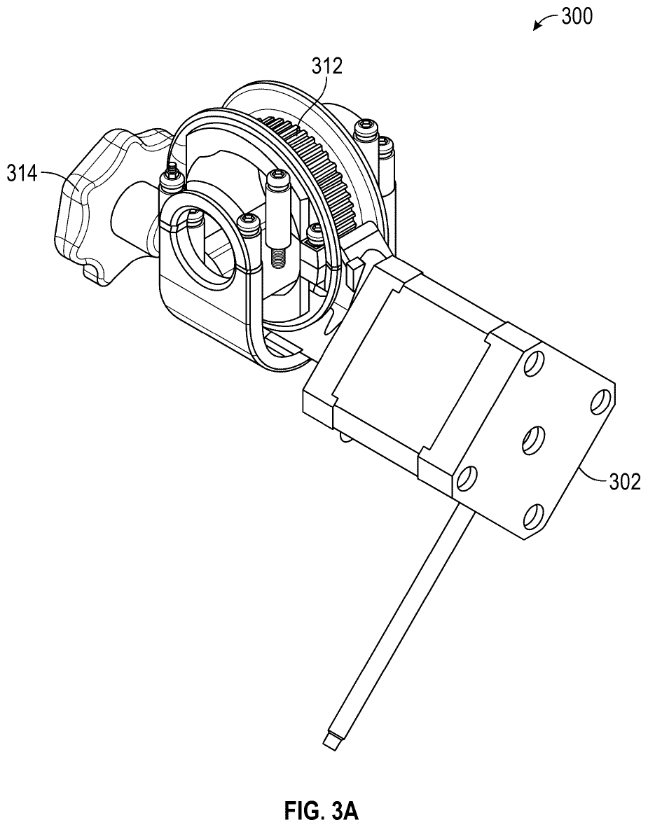

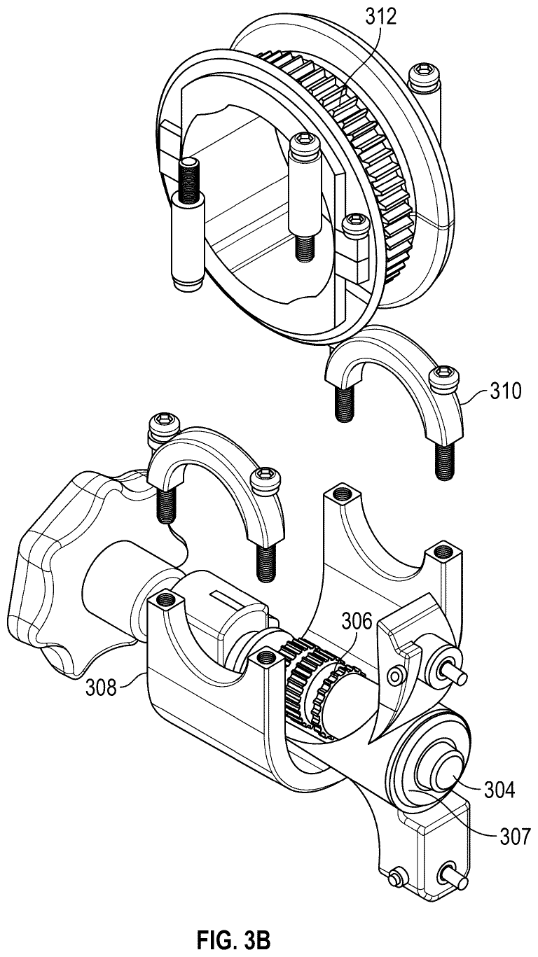

FIGS. 3A and 3B depict a drive mechanism 300 that can be fit on each of the outer guide catheter rotation knob 106, the flex controller 108, the inner catheter rotation knob 110, and the flex controller 112. The drive mechanism 300 is composed of a motor 302, for example a linear motor. The motor 302 connects to a shaft 304 on which is mounted a pinion gear 306 (e.g., a worm gear). The shaft 304 is supported by bearings 307 mounted in a carrier 308. The carrier 308 is configured to receive the catheter system 100, and clamps 310 secure the catheter system 100 to the carrier 308. A ring gear 312 is placed around and secured to the rotation knob 106, 110, or the flex controller 108, 112. By securing the ring gear 312 to the rotation knob 106, 110, or the flex controller 108, 112, the ring gear 312 cannot rotate relative to the rotation knob 106, 110, or the flex controller 108, 112. The exterior of the ring gear 312 includes teeth forming a rack that mates with the pinion gear 306. As a result, rotation of the motor 302 rotates the shaft 304 and therewith the pinion gear 306. Rotation of the pinion gear 306 is then transmitted to the teeth on the ring gear 312 and therewith effectuates rotation of rotation knob 106, 110, or the flex controller 108, 112 to achieve the articulation and rotation described above. Though a ring gear 312 and a pinion gear 306 are described herein, those of ordinary skill in the art will recognize that other forms of gearing can be utilized to achieve similar actuation (e.g., advancement or articulation) of the catheter.

For each drive mechanism 300 a manual rotation knob 314 connects to the shaft 304. As will be appreciated, rack and pinion gear sets, and particularly ones where a worm gear is employed as the pinion gear are not susceptible to being back driven without a suitable force being applied to the shaft on which the pinion gear is formed. As a result, following movement of the shaft 304 by the motor 302, the catheter system 100 will essentially lock in place and maintain its position. The rotation knob 314 allows for manual manipulation of the catheter system 100 and can overcome the friction of the pinion gear 306 on the ring gear 312 and allow for the pinion gear 306 to be forward driven or back driven enabling manual fine adjustment of the rotation or articulation of the relevant portion of the catheter system 100.

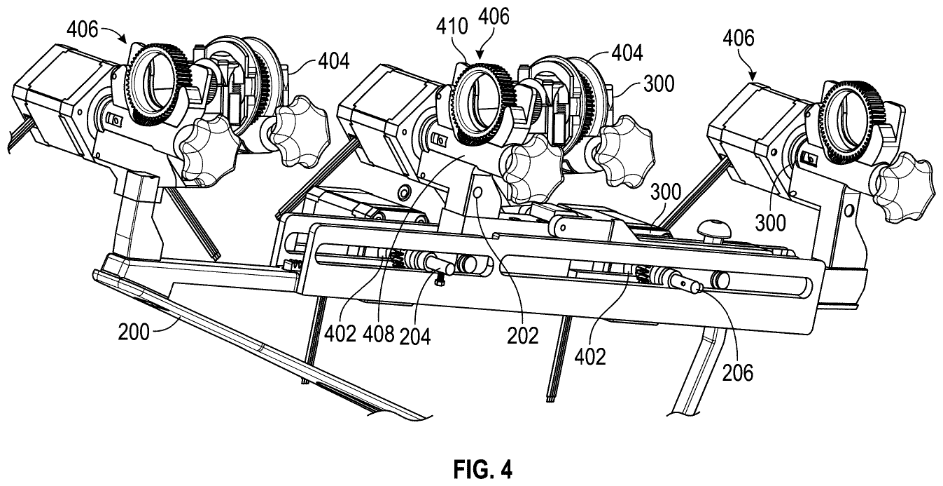

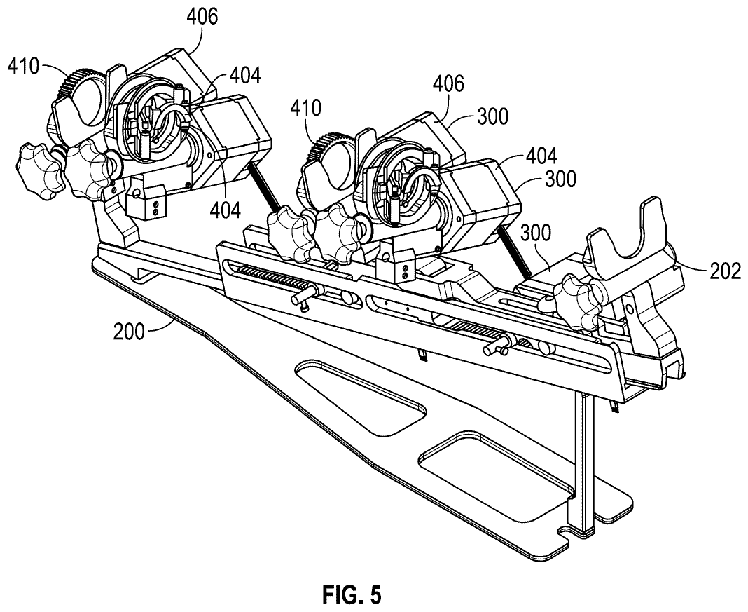

FIG. 4 depicts a front perspective view and FIG. 5 depicts a rear perspective view of drive mechanisms 300 mounted on the cradle 200 to drive the catheter system 100. There are three types of drive mechanisms 300 depicted in FIGS. 4 and 5. The first drive mechanism type 402 consists of a motor 302 which mounts directly to the shaft connected to the inner catheter drive knob 204 or the capsule catheter drive knob 206. The cradle 200 includes pinion gears mounted to the shafts (not shown) on which the inner catheter drive knob 204 or the capsule catheter drive knob 206 act. In this fashion the first type of drive mechanism 402 controls the movements of the inner catheter drive knob 204 or the capsule catheter drive knob 206.

A second type of drive mechanism 300 is drive mechanism 404 which is configured to be mounted to the outer guide catheter rotation knob 106 and the inner catheter rotation knob 110. The drive mechanisms 404 are identical to drive mechanism 300, as described above. For clarity, drive mechanism 404 is described and used in further description of this disclosure herein below. Through the use of the carrier 308 and clamps 310, the drive mechanism 404 can be attached to the catheter system 100 and is effectively supported by the catheter system 100 without being affixed to the cradle 200.

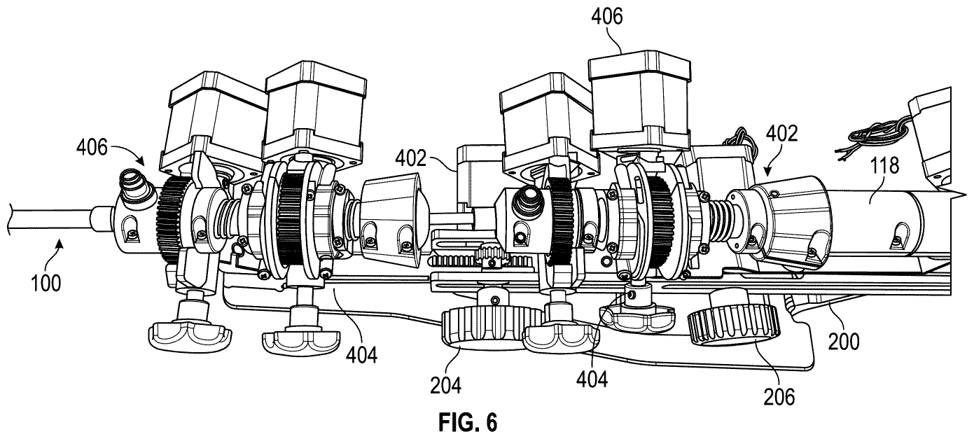

The third type of drive mechanism 300 is drive mechanism 406. Drive mechanism 406 is configured for connection to mate with flex controllers 108 and 112. The drive mechanism 406 includes a housing 408 that mounts on the supports 202 of the cradle. A motor 302 drives a shaft 304 that extends through the housing 408 where a manual rotation knob 314 is attached to the shaft 304. A pinion gear 306 (e.g., a worm gear) is mounted on the shaft 304. A ring gear 410 is mated to the flex controllers 108 or 112. The catheter 100, as described above, rests in and is secured to the supports 202 to which the housing 408 is secured. As a result the ring gear 410 can be secured to the flex controllers 108 and 112 of the catheter system 100 and then the catheter system 100 with the multiple drive mechanisms 300 attached can be set in and secured to the cradle 200 as shown in FIG. 6.

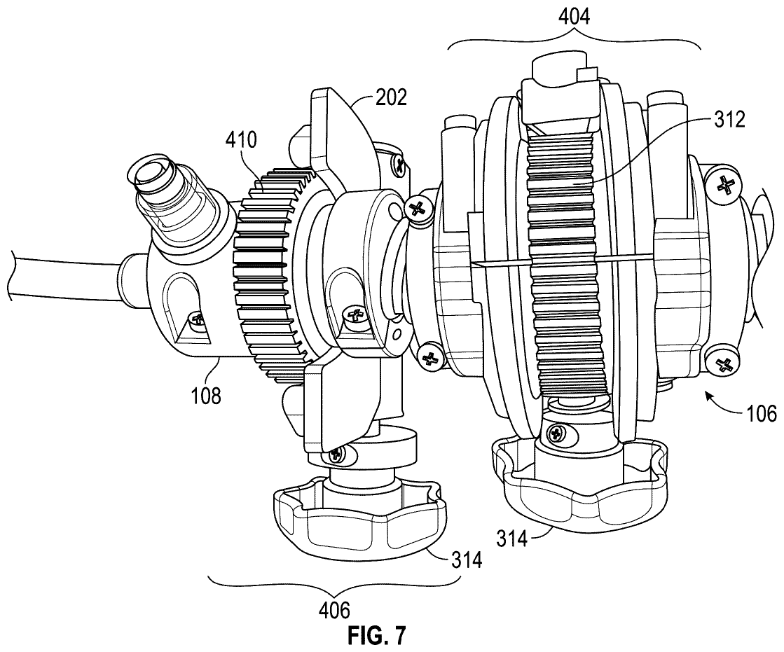

In some instances, a further drive mechanism 406 may be deployed as depicted in FIG. 7 at the proximal most portion of the cradle 200. Thus a total of 7 drive mechanisms may in some instances be utilized. The drive mechanism 406, may be optional employed to rotate the entirety of the catheter system 100 about its longitudinal axis. However, where such a drive mechanism is not employed, a user interface and controller (described below) may be utilized to ensure that drive mechanisms 404 operate simultaneously to achieve coordinated and synchronous rotation of the outer catheter 102 and the inner catheter 104.

FIG. 7 depicts a top view of a drive mechanism 406 and a drive mechanism 404 connected to a catheter system 100. The catheter system 100 rests on and is secured to the cradle 200. The ring gears 312 and 410 as well as the rotation knobs 314 connect to shafts 304 for manual rotation of the ring gears 312 and 410 are depicted as well.

Though not depicted in FIGS. 4-6, the cradle 200 and catheter system 100 may be configured such that the catheter system 100 and the cradle 200 are in a sterile environment. The catheter system 100 and cradle 200 may then have a sterile drape placed over them, providing a boundary between a sterile side and a non-sterile side. The housing 400 and carrier 308 along with the ring gears 312 and 410, and other components may all be sterile and assembled in a sterile environment prior to placement on the sterile cradle. The motors 302, which connect to each of the drive mechanisms 402, 404, 406, may then be attached to the cradle 200 and catheter system 100 from a non-sterile side. Thus, the motors 302 are not required to be sterilized since they do not come in direct contact with any component contacting the catheter system 100.

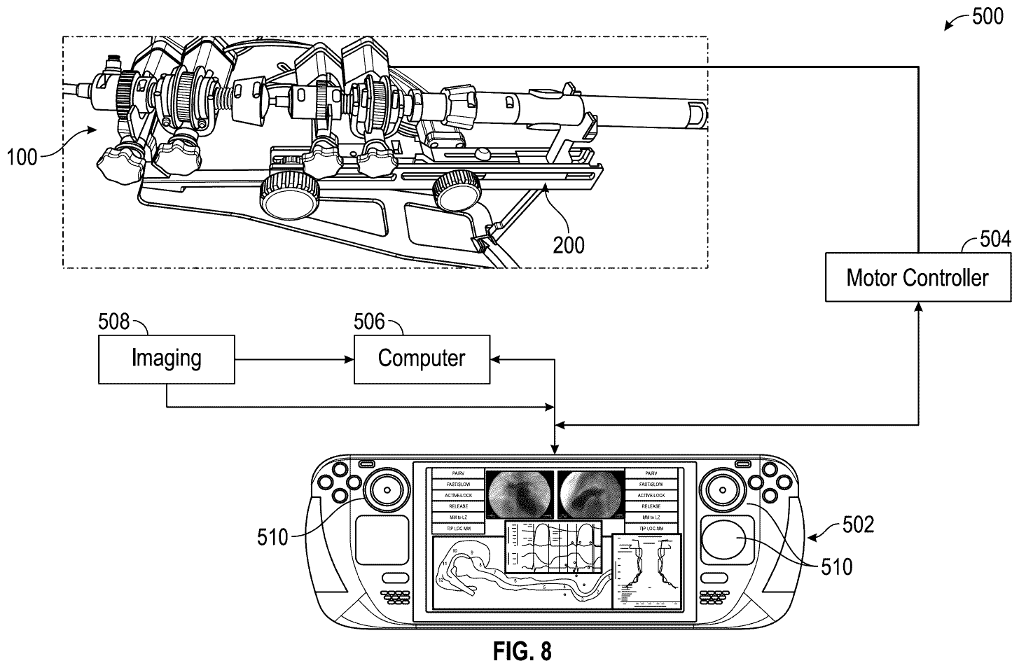

FIG. 8 depicts a complete mechanized catheter navigation system 500. The catheter navigation system 500 includes a catheter system 100 on which two drive mechanisms 404 are connected, and the cradle 200 on which the catheter system 100 is secured. The cradle 200 also supports drive mechanisms 402 and 406. The navigation system 500 includes a user interface 502 which is connected to the drive mechanisms 402, 404, and 406 via a motor controller 504. The user interface 502 may be connected to a computer 506, or may incorporate sufficient computing power (e.g., memory, processor, and software) to provide instructions to the motor controller 504. Also connected to the user interface 502 are one or more imaging modalities 508. The imaging modalities 508 may be ultrasound, fluoroscopy, computed tomography (CT), cone beam computed tomography (CBCT), magnetic resonance (MR), and others without departing from the scope of the disclosure. The imaging modalities 508 may be connected directly to the user interface 502 or connected via the computer 506. The computer also includes memory, processors, and software enabling interaction with the imaging modalities 508 and the user interface 502.

The user interface 502 includes a display 509. The display 509 is configured to present to the user a variety of data via a graphic user interface (GUI). In some aspects of the disclosure the display 509 is a touch screen display allowing input to be received directly through the display 509. The presented data may include imaging acquired from the imaging modalities 508, and data from one or more navigation programs (e.g., an electromagnetic (EM) navigation program) that updates the navigation of a distal portion of the catheter system 100 as it traverses the luminal network of the patient to a desired location within the patient. In addition, the display may present a workflow for a procedure (e.g., TMVR, lung navigation, etc.) whereby at one or more instances of the workflow guidance is provided to the user or input (e.g., selection of an option or identification of tissue) is requested from the user before advancing to the next portion of the workflow. In some instances, a navigation pathway through the luminal network of the patent may be determined from pre-procedural imaging (e.g., CT or MRI imaging). That navigation pathway may be registered to the position of the patient and advancement of the catheter system 100 within the patient may be presented in the GUI and provide positional information to the user without continuous imaging.

In practice, for a TMVR it is the final approximately 10 cm of navigation that is the most stressful for the surgeon and require the most care to prevent unintentional contact with the vasculature and the side wall of the left ventricle of the heart. As a result, the manipulations of the distal portion of the catheter system 100 must be carefully controlled. The motorized control of the outer catheter rotation knob 106, flex controller 108, inner catheter rotation knob 110, flex controller 112, inner catheter drive knob 204, and capsule catheter drive knob 206 allows for minute adjustments and control of particularly the distal portion of the catheter system 100. Mechanized actuation and control of these various knobs and controllers enables the catheter system to be quickly navigated, much faster than manual manipulation but with precise control.

The user interface 502 includes a number of inputs 512. Each input 510 may control two of the drive mechanisms 402, 404, and 406. As shown, each input 510 may be a joystick, however, the disclosure is not so limited, and inputs may be trackballs, wheels, and other inputs commonly employed in game controllers and other handheld input devices. Where joysticks are employed, a first joystick input 510 may control the motor 302 associated with the outer catheter rotation knob 106 when toggled in a first direction (e.g., up and down) and motor 302 associated with the flex controller 108 when toggled in a second direction (e.g., left and right). A second joystick input 510 may control the motor 302 associated with the inner catheter rotation knob 110 when toggled in a first direction (e.g., up and down) and motor 302 associated with the flex controller 112 when toggled in a second direction (e.g., left and right). A third joystick input 510 may control the motor 302 associated with the inner catheter drive knob 204 when toggled in a first direction (e.g., up and down) and control the motor 302 associated with the capsule catheter drive knob 206 when toggled in a second direction (e.g., left and right).

Through manipulation of the three inputs 510, signals are generated by the user interface 502 and directed to the motor controller 504. The motor controller 504 receives the signals generated by the user interface (e.g., digital signals) and converts the signals to a drive signal capable of powering the relevant motors 302 (e.g., an analog signal). Accordingly the motor controller 504 includes digital to analogue converters, a power source, and other power electronics capable of producing an output that drives the motors 302. The motor controller 504 may receive feedback from the motors 302 to confirm the drive of the motors 302 in conformance with the input received via the user interface 502. Where navigation software is employed, the change in position or orientation of the catheter system 100, particularly the distal portion of the catheter system 100 may be detected and a representation of the changes may be displayed in one or more of the windows of the GUI depicted on the display.

At various portions of a procedure imaging is acquired via the imaging modalities 508 (e.g., trans esophageal ultrasound, fluoroscopy, etc.). The images captured by the imaging modalities may be depicted in the GUI on the display 509. This placement of the images on the GUI in the display 509 immediately proximate the inputs 510 allow for close inspection by the surgeon and accurate finite control of the movements (e.g., flexure, rotation, advancement) of portions of the catheter system 100.

Still further, guidance regarding placement of a valve may be presented on the GUI in the form of a software generated silhouette of, for example, a deployed replacement mitral valve. The surgeon may then adjust the location of the silhouette on the GUI to optimize placement. Once the silhouette is placed in the GUI (e.g., on the live fluoroscopy images) the catheter system may be navigated, and the actual replacement valve (e.g., mitral valve) may be positioned for deployment within the patient.

Though described herein as a completed product with the drive mechanisms 402, 404, 406 connected to the catheter system 100, the disclosure is not so limited. In accordance with the disclosure drive mechanisms 402, 404, 406 may be offered as part of a kit for retrofitting existing catheter systems. The kit may optionally include the cradle 200. As will be appreciated the ring gears 312, 410 may be sized to fit rotating components of the existing catheter system. Alternatively, the kit may include a number of sizes of ring gears 312, 410, or the ring gears 312, 410 may have a variable sizing capability or a inserts provided therewith to enable attachment to various sizes of elements on the catheter system. Further, the cradle 308 and housing 408 may include variable clamps or attachment points for connection to various supports 202 of existing cradles designed for the catheter system.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules without departing from the scope of the disclosure.

We claim:

1. A system for navigation of a luminal network comprising:
a catheter including a distal portion configured for articulation and a handle;
a cradle configured to receive and support the handle;
a first motor connected to the catheter, wherein the motor is operable to articulate the distal portion of the catheter;
a second motor connected to the catheter, wherein the motor is operable to rotate the catheter about its longitudinal axis; and
a user interface including at least one input, wherein signals generated by manipulation of the input are delivered to the first motor to articulate the catheter and to the second motor to rotate the catheter.

2. The system for navigation of claim 1, wherein the cradle includes at least two supports configured to receive catheter.

3. The system for navigation of claim 2, further comprising a housing secured to one of the at least two supports, the housing including a shaft connected to the first motor, the shaft including a pinion gear.

4. The system for navigation of claim 3, further comprising a ring gear secured to a flex controller, the flex controller operable to cause the distal portion of the catheter to articulate.

5. The system for navigation of claim 4, wherein rotation of the flex controller articulates the distal portion of the catheter.

6. The system for navigation of claim 5, wherein the ring gear mates with the pinion gear such that rotation of the shaft and pinion gear results in rotation of the ring gear and the flex controller.

7. The system for navigation of claim 6, wherein the pinion gear is a worm gear.

8. The system for navigation of claim 1, further comprising a carrier including a shaft configured to mate with the second motor, and a pair of clamps securing the carrier to the catheter.

9. The system of navigation of claim 8, further comprising a ring gear secured to a catheter rotation knob, wherein the ring gear mates with a pinion gear on the shaft and rotation of the shaft by the motor results in rotation of the ring gear and the catheter rotation knob.

10. The system of navigation of claim 1, wherein the catheter includes an inner catheter, and outer catheter and a capsule catheter.

11. The system of navigation of claim 10, further comprising a third motor configured to mate with the cradle and effectuate longitudinal translation of the inner catheter relative to the outer catheter.

12. The system of navigation of claim 11, further comprising a fourth motor configured to mate with the cradle and effectuate longitudinal translation of the capsule catheter relative to the inner catheter and the outer catheter.

13. The system for navigation of claim 12, further comprising a motor controller configured to receive signals from the user interface and output signals to drive the first, second, third, and fourth motors.

14. The system for navigation of claim 12, further comprising a display on the user interface, wherein the display is configured to present a graphic user interface (GUI).

15. The system for navigation of claim 14, wherein the user interface is configured to receive images from one or more imaging modalities and display the images on the GUI.

16. The system for navigation of claim 1, wherein the catheter is a nested catheter a first catheter inside of a second catheter.

17. The system for navigation of claim 16, wherein the first catheter is connected to the first motor to articulate the distal portion of the first catheter.

18. The system for navigation of claim 16, wherein the first catheter is connected to the second motor to rotate the first catheter about its longitudinal axis.

19. The system for navigation of claim 16, wherein the second catheter is connected to a third motor to articulate the distal portion of the second catheter.

20. The system for navigation of claim 16, wherein the second catheter is connected to a fourth motor to rotate the second catheter about its longitudinal axis.
